(19) United States
(12) Patent Application Publication
(10) Pub. No.: US 2009/0130046 A1
(43) Pub. Date: May 21, 2009

(54) Concentrated Fragrance Composition Provided in Metered Aerosol Spray

(75) Inventor: Paul Alexander Clark, Racine, WI (US)

Correspondence Address: S.C. JOHNSON & SON, INC., 1525 HOWE STREET, RACINE, WI, 53403-2236, US

(73) Assignee: S.C JOHNSON & SON, INC., Racine, WI (US)

(21) Appl. No.: 11/943,051

(22) Filed: Nov. 20, 2007

(51) Int. Cl. A61L 9/012

(52) U.S. Cl. 424/76.2

(57) ABSTRACT

An aerosol fragrance composition for air freshening is disclosed, wherein the composition comprises a fragrance, one or more organic solvents, a propellant, and other optional ingredients. In use, the composition may be included in an aerosol device having a metered valve for delivering a predetermined dose to the air. Preferably, the aerosol fragrance composition is a single-phase composition so that the dispensing of the composition does not require vigorous shaking of the aerosol device before the composition is delivered to the air Disclosed compositions can be formulated to provide an equivalent or near equivalent performance when dispensed in 60 μL doses as with 120 μL doses, thereby providing a product with more doses per volume without compromising effectiveness.

### BACKGROUND

1. Technical Field

A concentrated fragrance aerosol composition for air freshening is disclosed. In use, the disclosed composition may be pressurized into an aerosol device to form a fragrance package. The aerosol device preferably includes a metered valve for precise dispensing of a predetermined amount of the fragrance composition to the air. Disclosed compositions can be formulated to provide an equivalent or near equivalent performance when dispensed in 60 μL doses as with 120 μL doses, thereby providing a product with more doses per volume without compromising effectiveness.

2. Description of the Related Art

Fragrances and perfumes contain molecules that diffuse via vaporization and activate one or more olfactory receptors in a user's nasal cavity. Often the level of fragrance needed in a product to achieve the desired response is relatively low This is particularly true for a fragrance that has a high vapor pressure at room temperature and atmospheric pressure, and that has a high odor activity.

One of the most popular uses of fragrances is air freshening, in which fragrance-containing compositions are delivered to the air either to treat or mask malodors, to create a pleasant and freshened impression to a user's sense of smell, or both. Malodors are typically organic molecules with different structures and functional groups, such as amines, acids, alcohols, aldehydes, ketones, phenols, polycyclics, indoles, aromatics, polyaromatics, etc. Malodors can be treated in a variety of ways, one of which is the masking of their presence with another, more pleasant odor, such as that of a fragrance.

Thus, odor masking can be defined as the intentional concealment of one odor (the malodor) with another (the fragrance). Odor masking may require large quantities of fragrance to ensure that the malodor is no longer noticeable, or is suitably masked. Further, as fragrances do not chemically modify or neutralize malodor molecules, odor masking techniques require stable and prolonged delivery of fragrance while the malodor is present

Numerous fragrance compositions and devices suitable for airborne delivery of fragrances have been developed in the art. Fragrance compositions may be in a form of a liquid spray or solid composite. A solid composite generally comprises a solid support, and a fragrance impregnated therein, wherein the solid support provides controlled and prolonged airborne release of the impregnated fragrance. The solid support may be a polymeric material such as a thermoplastic or an inert inorganic material that is compatible with the ingredients of the fragrance.

A more popular fragrance delivery mechanism is to spray a liquid composition to the air that needs to be freshened. One advantage of liquid fragrance sprays over solid fragrance composites is that a spray enables a user to control the amount and rate of the fragrance delivery. An aerosol device may be employed to dispense a fine mist of aerosol droplets. Aerosol devices may further include a metered valve for precise dispensing of predetermined doses of the liquid composition. Alternatively, a trigger, pump or other spray mechanism may be used as is known in the art

Because the particle sizes of aerosol droplets are significantly smaller than those of non-aerosol droplets, an aerosol fragrance composition can be delivered farther from the spray source, more evenly and aerosol droplets are less likely to “rain” out of the air than conventional pump spray compositions. Moreover, droplets of the aerosol fragrance compositions generally stay in the air for a longer period of time than those of non-aerosol compositions. As a result, the air freshening performance of an aerosol fragrance composition is generally better than that of a non-aerosol fragrance composition.

In general, aerosol fragrance compositions include at least one fragrance and at least one propellant, such as a conventional hydrocarbon propellant, to drive the compositions out of the aerosol devices. The compositions may further include one or more organic solvents to increase the solubility of the components therein.

An aerosol fragrance composition may be a single-phase composition, in which the fragrance is evenly distributed in the composition, or a multi-phase, in which the fragrance distribution varies. Generally, the single-phase composition is more desirable because a multi-phase composition requires a user to vigorously shake the aerosol device before the composition is dispensed. As a result, a multi-phase composition generally cannot be delivered by a continuous or automated aerosol device.

Known aerosol fragrance compositions sometimes contain too much fragrance. For example, one single-phase aerosol fragrance composition includes more than 55 wt % fragrance and 10-45 wt % propellant Such high concentrations of fragrance generally result in excessive sensory perception of the fragrance, which decreases the air freshening performance thereof

Hence, there is a need for a single-phase aerosol fragrance composition that includes a suitable amount of fragrance for air freshening purposes. Further, there is a need for an aerosol package that combines a single-phase aerosol fragrance composition with an aerosol device having a metered valve for precise dispensing of a predetermined amount of the composition to the air. Finally, there is still a need for a single-phase aerosol fragrance composition that is suitable for use in association with an aerosol device having a metered valve

### SUMMARY OF THE DISCLOSURE

An aerosol fragrance composition for air freshening is disclosed, wherein the composition comprises a fragrance, one or more organic solvents, a propellant, and other optional ingredients. In use, the composition may be included in an aerosol device having a metered valve for delivering predetermined doses of the composition to the air.

Preferably, the aerosol fragrance composition is a single-phase composition so that the dispensing of the composition does not require shaking of the aerosol device before the composition is delivered to the air “Single-phase,” as used herein, means the aerosol fragrance composition is homogeneous and substantially free of phase separation.

The fragrance used in the disclosed composition may be any fragrance or perfume suitable for air freshening purpose. A wide variety of strength and odor profile of the fragrance can be accommodated in the disclosed composition as long as the fragrance is chemically compatible with other components of the composition and the ingredients included therein do not affect the single-phase presence of the composition

Preferably, the amount of the fragrance included in the disclosed composition enables effective air freshening for an extended period of time with each dose dispensed by the metered valve of the aerosol device In one embodiment, the disclosed composition comprises from about 8 to about 20 wt % fragrance.

The disclosed composition may be combined with an aerosol device into an aerosol fragrance package. The aerosol device may be any device known in the art that is capable of storing the composition under pressure and dispensing the composition through a nozzle as a mist of fine aerosol droplets.

The aerosol device may comprise a metered valve for precise dispensing of doses of predetermined doses of the composition to the air. The metered valve may be of any structure, design or material known in the art. The amount of the composition included in each dose generally depends on factors such as the amount of the air that needs to be freshened, the strength and odor profile of the fragrance, etc In one embodiment, the metered valve is a 60 μL valve The device may be actuated either automatically by a programmable actuator or manually by a user.

In a refinement, the composition is formulated so that the Fragrance Strength as defined below of a disclosed composition dispensed through a 60 μL metered aerosol device is no less than 80% of the Fragrance Strength of the composition dispensed through a 120 μL metered aerosol device.

In another refinement, the composition is formulated so that the Fragrance Acceptance as defined below of a disclosed composition dispensed through a 60 μL metered aerosol device is no less than 80% of the Fragrance Acceptance of the composition dispensed through a 120 μL metered aerosol device.

In a further refinement, the composition is formulated so that both the Fragrance Acceptance and Fragrance Strength as defined below of a disclosed composition dispensed through a 60 μL metered aerosol device are no less than 80% of the Fragrance Acceptance and Fragrance Strength of the composition dispensed through a 120 μL metered aerosol device.

According to another aspect of this disclosure, a solvent system may be included in the aerosol fragrance composition for solubilizing the fragrance and other ingredients included therein. Preferably, the inclusion of the solvent system does not affect the single-phase presence of the aerosol composition. In one embodiment, the concentration of the solvent system in the aerosol fragrance composition is from about 25 to about 50 wt %.

The solvent system may comprise any organic solvent or mixtures thereof suitable for use in an aerosol composition. In one embodiment, the organic solvent system comprises one or more monohydric alcohols, preferably low molecular weight monohydric alcohols such as ethanol, propanol, isopropanol, and butanol.

Some low-molecular weight polyhydric alcohols, such as glycols, may also be included in the organic solvent system Further, the solvent system may comprise one or more non-alcoholic organic solvents, such as acetone, C5-C9 hydrocarbon solvents, esters and diesters.

In addition to the above discussed organic solvent, the solvent system of the disclosed composition may optionally include a small amount of water. In one embodiment, the disclosed composition comprises no more than about 10 wt % water When water is present in the composition, the composition may further optionally include one or more corrosion inhibitors to prevent corrosion of the aerosol container or metallic parts of the aerosol device.

In order to drive the disclosed composition out of the aerosol device, a propellant is included in the composition. The propellant may be any conventional propellant known in the art that is compatible with the fragrance and other ingredients of the composition. In one embodiment, the aerosol fragrance composition comprises from about 30 to about 67 wt % propellant

Preferably, the inclusion of the propellant does not affect the single-phase presence of the aerosol fragrance composition. Suitable propellants include, but are not limited to, hydrocarbons, halogenated hydrocarbons, ethers, carbon dioxide, compressed air, compressed nitrogen, etc. In one refinement, the propellant is a B-60 propellant, which is a mixture of propane, butane and isobutane. In another refinement, the propellant is an A-60 propellant, which is a mixture of propane and isobutane.

It is unexpected that the combination of the fragrance, solvent system and propellant disclosed herein in a single-phase composition provides a more desirable fragrance concentration in the aerosol composition without affecting its single-phase presence and, as a result, renders the aerosol composition more suitable for use in metered doses. Specifically, test data indicates that the disclosed compositions are advantageously dispensed using a 60 μL valve instead of a 120 μL as approval ratings for 120 μL doses were not significantly higher than those for the 60 μL doses.

More importantly, as the disclosed composition maintains a single-phase presence, improved air freshening performance is achieved without the drawbacks of a multi-phase aerosol formulation that requires shaking before each application.

A method for using the disclosed composition for air freshening is also disclosed. The method comprises providing the aerosol fragrance package described above and dispensing the composition to the air through a metered valve such as a 60 μL metered valve, wherein the composition imparts freshening impression to the air.

Because the disclosed aerosol fragrance composition is preferably a single-phase composition that does not require shaking or mixing prior to use, the composition can be utilized in an automated system that dispenses the formulation continuously, periodically or at timed intervals.

Other advantages and features of the disclosed embodiments and methods will be described in the following detailed description of the presently preferred embodiments.

### BRIEF DESCRIPTION OF THE DRAWINGS

For a more complete understanding of the aerosol fragrance composition and air-freshening package, reference should be made to the embodiments illustrated in greater detail in the accompanying drawing, wherein:

FIG. 1 is a front sectional view of an aerosol device suitable for use in association with the disclosed aerosol fragrance composition accordance this disclosure.

It should be understood that the drawing is not necessarily to scale and that the disclosed metered valve embodiment is illustrated diagrammatically and in a partial view. In certain instances, details which ate not necessary for an understanding of the disclosed apparatus or which render other details difficult to perceive may have been omitted. It should be understood, of course, that this disclosure is not limited to the particular embodiments illustrated herein

### DETAILED DESCRIPTION OF THE PRESENTLY PREFERRED EMBODIMENTS

In this disclosure, the terms “fragrance” and “perfume” will be used interchangeably and the collective singular terms, fragrance and perfume, may mean one or more fragrant ingredients.

In general, this disclosure is directed toward an air-freshening aerosol fragrance composition comprising a fragrance, one or more organic solvents, and a propellant The composition may further include optional ingredients such as water, corrosion inhibitors, deodorizing agents, antimicrobial agents, preservatives, and the like.

The aerosol fragrance composition is preferably a single-phase liquid composition so that the air freshening performance of the composition may be significantly improved without sacrificing the convenience provided by the single-phase product Without being bound by any particular theory, it is contemplated that, the combination of the fragrance, organic solvent(s) and propellant in the disclosed concentration ranges results in an equilibrium state in which the composition maintains a homogeneous, single-phase presence.

In a general embodiment, the disclosed aerosol fragrance composition comprises from about 8 to about 20 wt % fragrance, from about 25 to about 50 wt % organic solvent, and from about 30 to about 67 wt % propellant. The aerosol formulation preferably has a single-phase presence at room temperature so that no shaking or mixing is required prior to its application.

Fragrance

The disclosed aerosol composition comprises a fragrance for air freshening. The fragrance freshens air either by masking one or more malodors therein and by imparting a pleasant smell to the air, or both. As is well known, a fragrance normally includes a mixture of a number of fragrant materials and auxiliary solvents. Exemplary auxiliary solvents include, but are not limited to: monohydric alcohols such as ethanol, propanol and isopropanol; glycols such as propylene glycol and dipropylene glycol; esters such as isopropyl myristate and isopropyl palmitate; and diesters such as dimethyl adipate, diisopropyl adipate and dibutyl adipate. The number of fragrant materials in a fragrance is typically ten or more. For the purpose of this disclosure, the fragrance used in the disclosed aerosol composition includes at least 10 wt %, more preferably at least 25 wt %, and most preferably at least 50 wt % fragrance materials

The range offiagrant materials used may vary. The materials may vary in chemical class, but in general are water-insoluble oils In many instances, the molecular weight of a fragrance material is in excess of 150, but does not exceed 300. However, fragrances with average molecular weights outside this range may be utilized as well.

The fragrance included in the disclosed aerosol composition may be present in an amount that is sufficient to impart a pleasant smell to the air that can be perceived by a consumer. In the presence of a malodor, the fragrance included in the aerosol composition may be present in an amount that masks at least a substantial portion of the malodor in the air. More preferably, the perfume included in the aerosol formulation is preferably present in an amount that not only completely masks the malodors therein, but also delivers a pleasant smell to be perceived by a consumer.

In one embodiment, the fragrance is present in the aerosol formulation in an amount of from about 8 to about 20 wt %, more preferably from about 8 to about 15 wt % and most preferably from about 8 to about 10 wt %. The amount of the fragrance that is needed to mask the malodor(s) therein, and/or the amount of the fragrance to impart the pleasant smell to be perceived by the consumer will be apparent to one of ordinary skill in the art.

The concentration of the fragrance and/or the dose of the composition dispensed to the air also depend upon the placement or application of the aerosol device and whether or not the device is an automated dispenser It is to be understood that, although fragrance amounts higher than about 20 wt % or lower than about 8 wt % can be accommodated in the disclosed aerosol composition, the concentration of the fragrance in the composition should be consistent with its air freshening application, particularly when delivered in metered doses.

More specifically, the inclusion of the fragrance at higher concentrations generally results in the inclusion of a very small volume of the composition in each metered dose. Such small doses require the use of a significantly smaller metered valve, which may present unnecessary challenges to the design and manufacturing of the aerosol device Similarly, the inclusion of the fragrance at lower concentrations not only requires the use of a significantly larger metered valve, but also depletes the composition at a much faster rate because more composition needs to be dispensed to achieve satisfactory air freshening result.

The fragrance according to this disclosure may comprise one or more fragrant materials or materials that provide chemically active vapors In one embodiment, the fragrance can comprise and/or include volatile, fragrant compounds including, but not limited to natural botanic extracts, essences, fragrance oils, synthetic fragrant materials and so forth As is known in the art, many essential oils and other natural plant derivatives contain large percentages of highly volatile scents. In this regard, numerous essential oils, essences, and scented concentrates are commonly available from companies in the fragrance and food businesses.

Exemplary oils and extracts include, but are not limited to, those derived from the following plants: almond, amyris, anise, armoise, bergamot, cabreuva, calendula, canaga, cedar, chamomile, coconut, eucalyptus, fennel, jasmine, juniper, lavender, lemon, orange, palm, peppermint, quassia, rosemary, thyme, and so forth.

In addition to natural extracts and oils, the fragrance used in the disclosed composition can be synthesized by those skilled in the art in a wide variety of odor profiles and strengths. Typical synthetic fragrances include one or more fragrant components including, but are not limited to, volatile phenolic substances (such as isoamyl salicylate, benzyl salicylate, and thyme oil red); aldehydes and ketones (such as B-methylnaphthyl ketone, p-t-butyl-A-methyl hydrocinnamic aldehyde and p-t-amyl cyclohexanone); polycyclic compounds (such as coumarin and naphthyl methyl ether); and esters (such as diethyl phthalate, phenylethyl phenylace-tate, non-anolide-1:4).

Fragrances can also be made of organic compounds derived from floral materials and fruits. Examples of suitable organic compounds include, but are not limited to, dimyrcetol, phenylethyl alcohol and tetrahydromuguol, decyl aldehyde, undecyl aldehyde, undecylenic aldehyde, lauric aldehyde, amyl cinnamic aldehyde, ethylmethyl phenyl glycidate, methyl nonyl acetaldehyde, myristic aldehyde, nonalactone, nonyl aldehyde, octyl aldehyde, undecalactone, hexyl cinnamic aldehyde, benzaldehyde, vanillin, heliotropine, camphor, parahydroxyphenolbutanone, 6-acetyl-1,1,3,4,4,6-hexamethyl tetrahydronaphthalene, alpha-methyl ionone, gamma-methyl ion-one, and amyl-cyclohexanone and mixtures thereof.

It is to be understood, of course, that the type, strength, and odor profile of the fragrance suitable for use in the disclosed aerosol composition would be apparent to one of ordinary skill in the art and therefore should not be considered as limiting the scope of this disclosure

Fragrance oils may be obtained from a variety of sources including Quest International (recently acquired by Givaudan S. A.; www.givaudan.com) of the Netherlands, Takasago International of Japan (www.takasago.com) and Firmenich International S.A. of Geneva Switzerland (www.firmenich.com). Other fragrance suppliers are well known to those skilled in the art.

Organic Solvent

In older to solubilize the fragrance and other ingredients, one or more organic solvents are included in the disclosed aerosol fragrance composition. Suitable organic solvents include monohydric alcohols, polyhydric alcohols, acetone, hexane, and other conventional organic solvent known in the art

In one embodiment, the organic solvent is selected from the group consisting of monohydric alcohols, glycols, acetone, C5-C9 hydrocarbon solvents, esters, diesters, and mixtures thereof The monohydric alcohols are preferably low molecular weight monohydric alcohols such as ethanol, propanol, isopropanol, butanol, isobutanol, etc. The glycols include, but are not limited to, ethylene glycol, propylene glycol, diethylene glycol, dipropylene glycol, triethylene glycol, etc. The most preferred organic solvent is ethanol due to its low cost and availability.

The disclosed aerosol fragrance composition may comprise from about 25 to about 50 wt %, more preferably from about 30 to about 45 wt %, and most preferably from about 35 to about 40 wt %, organic solvent(s). In one embodiment, the disclosed composition comprises about 40 wt % ethanol.

As shown in Examples 1-9 below, a wide range of solvent types and concentrations may be accommodated within the scope of this disclosure. Further, the inclusion of other organic solvents at appropriate concentrations will be apparent to one of ordinary skill in the art without undue experimentation.

Propellant

Aerosol propellant is an essential element in any aerosol composition as it provides constant pressure for dispensing the composition through the nozzle. For commercial aerosols, the propellant or propellant mixture will typically have a boiling point slightly lower than room temperature. As a result, inside the pressurized can, the vapor phase of the propellant exists in equilibrium with the liquid phase of the propellant at a vapor pressure that is higher than atmospheric pressure, and as a result, the vapor phase of the propellant is able to drive the composition out of the can when the nozzle is opened. Moreover, as the vapor phase of the propellant escapes through the nozzle, it is immediately replenished by evaporation of the liquid phase of the propellant as equilibrium within the can is reestablished

The propellant that is suitable to be used in the aerosol fragrance composition according to this disclosure may be selected from the group consisting of hydrocarbon propellants, ether propellants, HFCs, CFCs, soluble or non-soluble compressed gases, and mixtures thereof. In one embodiment, the propellant suitable for use in the disclosed aerosol composition is selected from the group consisting of propane, isobutane, n-butane, dimethyl ether, HFC-152a (1,1-difluoroethane), and mixtures thereof. The preferred propellant according to this disclosure may include one or more hydrocarbon propellants In one embodiment the propellant is a mixture of propane, isobutane and n-butane Other propellants or propellant mixtures that may be included in the disclosed aerosol composition will be apparent to those skilled in the art

The propellant may be present in the aerosol fragrance composition in a wide range of concentrations, as demonstrated in Examples 1-9 below Generally, the propellant of the disclosed composition is the balance of the fragrance, organic solvent(s), and other additional ingredients. In one embodiment, the composition comprises from about 30 to about 67 wt %, more preferably from about 40 to about 60 wt %, and most preferably from about 45 to about 55 wt %, propellant. It is noteworthy that the propellant may also function as a solvent to facilitate the solubilization of the fragrance.

In a more specific embodiment, the aerosol composition comprises about 51 wt % of a B-60 hydrocarbon propellant, which is a mixture of 44% propane, 23% isobutane, and 32% n-butane. In another embodiment, the aerosol composition may include an A-60 hydrocarbon propellant, which is mixture of 70% propane and 30% isobutane. Further, the inclusion of the propellant at other appropriate concentrations will be apparent to one of ordinary skill in the art without undue experimentation.

Optional Ingredients

As discussed above, the disclosed composition may optionally include one or more additional ingredients such as water, corrosion inhibitor, deodorizing agents, emulsifiers, and the like. Up to about 10 wt % water can be included in the composition to possibility lower the flammability of the composition and/or to decrease the cost of its manufacturing.

However, when water is included in the disclosed composition the potential risk of corrosion on the interior of the aerosol device increases if the device is manufactured of a metallic material without anticorrosion treatment, thus leading to contamination of the aerosol fragrance composition and ultimately to leaking of the aerosol device if the corrosion is severe enough. In such cases, corrosion inhibitors are preferably included in the disclosed composition.

There are many corrosion inhibitors available in the art. In one embodiment, the corrosion inhibitor used in the disclosed composition is selected from the group consisting of potassium phosphates, potassium nitrite, sodium phosphates, sodium nitrite, ammonium phosphates, ammonium nitrite, triethanolamine, sodium benzoate, borax (Na₂B₄O₇H₂O), and mixtures thereof. The type and concentration of the corrosion inhibitor suitable for use in the aerosol fragrance composition would be apparent to one of ordinary skill in the art and should be considered as within the scope of this disclosure

The disclosed composition may also include a deodorizing agent to eliminating the malodors or odor-causing microorganisms in the air, thereby further increasing the odor freshening performance of the composition. Suitable deodorizing agents include, but are not limited to, glycols such as triethylene glycol and dipropylene glycol, triethanolamine, cyclodextrins and derivatives thereof, oxidizing agents, reducing agents, peroxides, and other deodorizing agents known in the art. Again, the type and amount of the deodorizing agent suitable for use in the disclosed composition would be apparent to one of ordinary skill in the art without undue experimentation.

The aerosol fragrance composition according to this disclosure may also include an emulsifier. The emulsifier may comprise one of more surfactants. There are many types of surfactants that can be included in the composition which include, but are not limited to, one or more cationic surfactants, anionic surfactants, nonionic surfactants, amphoteric surfactants, and mixtures thereof. In general, the emulsifier is included in the composition at a concentration of 0-4 wt %.

It is to be understood, of course, that the inclusion of the optional ingredient(s) in the disclosed composition preferably does not affect the single-phase presence of the composition.

Without being bound by any particular theory, it is believed that an enhanced air freshening performance is provided by the single-phase aerosol fragrance composition disclosed herein Another benefit of the single-phase aerosol formulation is the provision of a desirable fragrance concentration that is not available in existing aerosol fragrance compositions. Still further, the single-phase aerosol formulation disclosed herein does not require shaking prior to application and therefore can be utilized in stationary (wall-mounted) and/or automatic systems and dispensers

Several non-limiting exemplary aerosol fragrance compositions (1-9) according to this disclosure are listed below. The fragrances used in those compositions are of different scent variations, and are all commercially available and used as received from the manufacturer.

### EXAMPLE 1

| | Description/ | |
|---|---|---|
| wt % | Function | Chemical/Trade Name |
| 20% | fragrance | TAKASAGO ™ RL 1610/1 |
| 55% | propellant | B-60 hydrocarbon |
| | | (propane/isobutane/n-butane) |
| 25% | solvent | ethanol |
| 100% | | |

### EXAMPLE 2

| | Description/ | |
|---|---|---|
| wt % | Function | Chemical/Trade Name |
| 15% | fragrance | TAKASAGO ™ RL 1610/1 |
| 46 8% | propellant | B-52 hydrocarbon (propane/n- |
| | | butane) |
| 8% | solvent | deionized water |
| 30% | solvent | ethanol |
| 0.02% | corrosion | KH₂PO₄, mono-potassium |
| | inhibitor | phosphate |
| 0.18% | corrosion | K₂HPO₄, di-potassium |
| | inhibitor | phosphate |
| 100% | | |

### EXAMPLE 3

| | Description/ | |
|---|---|---|
| wt % | Function | Chemical/Trade Name |
| 10% | fragrance | TAKASAGO ™ RO 3601/2 |
| 40% | propellant | B-52 hydrocarbon (propane/n- |
| | | butane) |
| 50% | solvent | ethanol |
| 100% | | |

### EXAMPLE 4

| | Description/ | |
|---|---|---|
| Wt % | Function | Chemical/Trade Name |
| 8% | fragrance | Firmenich SJ150 362/G |
| 52% | propellant | B-60 hydrocarbon |
| | | (propane/isobutane/n-butane) |
| 40% | solvent | ethanol |
| 100% | | |

### EXAMPLE 5

| | Description/ | |
|---|---|---|
| wt % | Function | Chemical/Trade Name |
| 20% | fragrance | Firmenich SJ150 434 |
| 50% | propellant | B-60 hydrocarbon |
| | | (propane/isobutane/n-butane) |
| 30% | solvent | ethanol |
| 100% | | |

### EXAMPLE 6

| wt % | Description | Chemical/Trade Name |
|---|---|---|
| 12% | fragrance | Firmenich SJ224 003 |
| 50% | propellant | B-60 hydrocarbon |
| | | (propane/isobutane/n-butane) |
| 30% | solvent | ethanol |
| 8% | solvent/deodorizing | triethylene glycol |
| | agent | |
| 100% | | |

### EXAMPLE 7

| wt % | Description | Chemical/Trade Name |
|---|---|---|
| 8% | fragrance | IFF Lillian 95 DPD Mod 2 |
| 67% | propellant | B-60 hydrocarbon |
| | | (propane/isobutane/n-butane) |
| 15% | solvent | ethanol |
| 10% | solvent | propanol |
| 100% | | |

### EXAMPLE 8

| wt % | Description/Function | Chemical/Trade Name |
|---|---|---|
| 20% | fragrance | Quest F557919 |
| 30% | propellant | A-60 hydrocarbon |
| | | (propane/isobutane) |
| 50% | solvent | ethanol |
| 100% | | |

### EXAMPLE 9

| wt % | Description/Function | Chemical/Trade Name |
|---|---|---|
| 18% | fragrance | Quest Q31020 |
| 54% | propellant | A-60 hydrocarbon |
| | | (propane/isobutane) |
| 28% | solvent | ethanol |
| 100% | | |

### Aerosol Device

As discussed above, the aerosol fragrance composition may be combined with an aerosol device to form an air-freshening package. Preferably, the aerosol device includes a metered valve member for precise dispensing of the disclosed composition in predetermined doses. In use, the composition may be automatically dispensed to the air at predetermined time intervals by an automatic actuator included in the aerosol device. Alternatively, a user can manually dispense the composition by pressing a manual actuator located at the top of the aerosol device. In either case, no shaking is needed before the dispensing of the composition because the composition has a single-phase presence

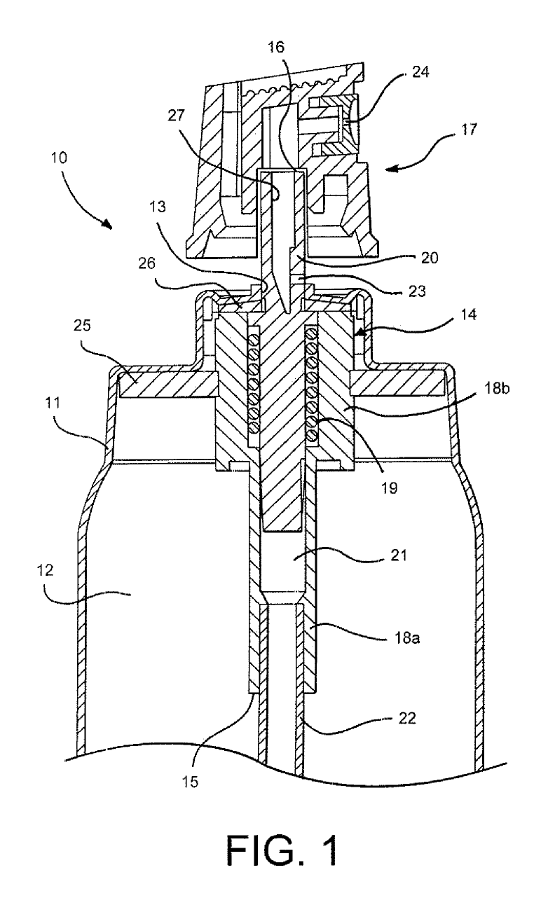

FIG. 1 graphically illustrates a non-limiting exemplary embodiment of an air-freshening package 10 comprising a housing 11 defining an internal cavity 12 and a top opening 13, and a metered valve member 14 including a bottom end 15 that is inserted into the internal cavity 12 and a top end 16 that extends out of the housing 11 through the opening 13. An actuator 17 is connected to the top end 16 of the valve member 14. The internal cavity 12 is charged with a disclosed composition under pressure using an apparatus and method well known in the art.

The metered valve member 14 further comprises a lower tubular housing 18a, an upper tubular hosing 18b, a spring 19 accommodated in the upper housing 18b, and an upper stem 20, wherein the lower tubular valve housing 18a defines a volumetric chamber 21 for metering the aerosol fragrance composition. The spring 19 and stem 20 are supported by the upper housing 18b which is held in place by a collar 25. A dip tube 22 is connected to the bottom end 15 of the metered valve member 14 which provides a flow channel for delivery of the aerosol fragrance composition from the internal cavity 12 to the volumetric chamber 21. The top end 16 of the metered valve member 14 is mateably received within the actuator 17 for pressing the stem 20 downward until the orifice 23 is disposed below the seal 26, thereby delivering the composition from the volumetric chamber 21 through the passageway 27 to the actuator 17.

The spring 19 and stem 20 may be constructed as separate or integrated parts. The spring 19 provides an energy constant to return the stem 20 to a closed/sealed position, as illustrated in FIG. 1. In the closed/sealed position of FIG. 1, the stem 20 and seal 26 shuts off the fluid communication between the volumetric chamber 21 and the stem orifice 23, but at the same time allows the flow of the composition from the dip tube 22 to the volumetric chamber 21.

When the aerosol fragrance composition needs to be dispensed, a user presses actuator 17 downward, thereby forcing the stem 20 to move downward until the bottom of the stem 20 engages the top of the dip tube 22. Such engagement shuts off the flow of the composition from the dip tube 22 to the volumetric chamber 21 but at the same time enables fluid communication from the volumetric chamber 21, through the orifice 23, through the passageway 27 and to the nozzle 24 of the actuator 17. As a result the amount of the composition that is charged in the volumetric chamber 21 is dispensed to the air through the spray nozzle 24.

The disclosed composition may be dispensed in many dose forms by providing metered valve members having different volumetric chamber capacities. For example, the volumetric chamber of the metered valve member can have a capacity of from 20 μL to 185 μL In a preferred embodiment, the aerosol device has a 60 μL metered valve member. In another embodiment, the aerosol device has a 120 μL metered valve member.

Air Fragrancing Performance

To evaluate the performance of the disclosed composition, fragrance evaluation tests were conducted based on olfactory perception of the fragrance aerosol spray according to this disclosure. In each of those tests, a single dose of one disclosed aerosol fragrance composition was delivered into the air of a test chamber After the lapse of a predetermined amount of time, an evaluator was allowed to sniff the air within the test chamber and evaluate the strength and acceptance of the fragrance The tested compositions included different concentrations of fragrances and were delivered into the test chamber by an automatic aerosol dispenser having either a 60 μL or 120 μL metered valve.

In the evaluation tests, three fragrance concentrations were chosen within the disclosed range: 8 wt %, 12 wt %, and 16 wt %. And four commercial fragrances were used to prepare the test fragrance compositions: S1, S2, S3, and S4.

The evaluation tests were conducted as blind balanced block monadic sequential tests as follows: (1) recruiting at least 80 female evaluators with ages between 18-55, each evaluator being exposed to instant action air freshener in the past six months; (2) preparing a plurality of identical enclosed test chambers; (3) dispensing different sample aerosol spray (in single doses) to each of those chambers; (4) within 30 minutes, allowing the evaluator to evaluate the strength or perception of the fragrance spray by sniffing the air in the test chambers (order of presentation was randomized to avoid a position bias); and (5) asking the evaluator to record their evaluation by responding to the questionnaire below.

Sample Questionnaire
(a). How do you feel about this fragrance?

1=Dislike Strongly, 9=Like Strongly
(b). How appropriate is this fragrance for an air freshener?

1=Not appropriate; 9=Very appropriate
(c). How strong is this fragrance?

1=Weak; 9=Strong
(d). How do you feel about the fragrance strength?

(1) Too weak; (2) Just about right; (3) Too strong

The average score of question (c) is defined as Fragrance Strength (FS), and the percentage of evaluator who responded to question (d) as “Just about right” is defined as Fragrance Acceptance (FA). Two important performance parameters of the disclosed fragrance composition are FA (0-10 min) and FA (21-30 minutes), which are the Fragrance Acceptance evaluated 0-10 or 21-30 minutes after the composition is delivered into the air. The Fragrance Acceptance of test compositions S1, S2, S3, and S4 at various fragrance concentrations evaluated at 0-10 min, 11-20 min, and 21-30 min, are listed at Table 1 below. All test compositions were delivered through a 60 μL automatic aerosol device.

TABLE 1
Fragrance Acceptance of Fragrance Compositions

| Fragrance | S1/8% | S2/8% | S3/8% | S4/8% | S1/12% | S2/12% | S3/12% | S4/12% | S3/16% | S4/16% |
|---|---|---|---|---|---|---|---|---|---|---|
| FA (0-10 min) | 57% | 58% | 58% | 47% | 59% | 56% | 33% | 52% | 42% | 54% |
| FA (11-20 min) | 42% | 77% | 63% | 31% | 72% | 57% | 45% | 43% | 52% | 35% |
| FA (21-30 min) | 46% | 60% | 56% | 56% | 54% | 50% | 50% | 54% | 48% | 56% |

Table 1 clearly indicates that the disclosed fragrance compositions are generally well accepted by the evaluators at all tested concentrations. Specifically, all tested compositions were perceived as “just about right” by more than 30% of the evaluators within 30 minutes after the compositions are dispensed into the test chambers. Surprisingly, the acceptance of fragrance strength does not significantly decrease more than 20 minutes after the fragrance is dispensed. In some cases, the test composition even exhibits an increased FA. In one embodiment, FA (21-30 min) of the disclosed composition is no less than 80% of FA (0-10 min) of the same composition.

To evaluate the effect of the dispensing device on the acceptance and strength of the disclosed composition, a comparative evaluation is conducted when two identical fragrance compositions S2 with containing 8 wt % fragrance were dispensed into two test chambers. One composition was dispensed through an automatic aerosol device having a 60 μL valve, while the other composition was dispensed through a similar device but having a 120 μL valve The resulting Fragrance Strength (FS) and Fragrance Acceptance (FA) are listed in Table 2 below

TABLE 2
Effect of Dose on Fragrance Strength and Fragrance Acceptance

| Fragrance/valve | S2 (8%)/60 μL | S2 (8%)/120 μL |
|---|---|---|
| FS (0-10 min) | 49 | 5.9 |
| FA (0-10 min) | 65% | 73% |

Again, it is unexpected that the increasing of dose does not substantially affect the Fragrance Strength (FS) and Fragrance Acceptance (FA) as defined above for the dispensed fragrance composition according to this disclosure In one embodiment FS (0-10 min) of the disclosed composition dispensed through a 60 μL device is no less than 80% of FS (0-10 min) of the same composition dispensed through a 120 μL device. In another embodiment, FA (0-10 min) of the disclosed composition dispensed through a 60 μL device is no less than 80% of FA (0-10 min) of the same composition dispensed through a 120 μL device Thus, disclosed compositions can be formulated to provide an equivalent or near equivalent performance when dispensed in 60 μL doses as with 120 μL doses, thereby providing a product with more doses per volume without compromising effectiveness

Without being bound by any particular theory, it is contemplated that the above unexpected results are caused by the type of propellant used in the disclosed composition, the concentrations of the fragrance, propellant amount, and solvent amount in the disclosed composition, and the single-phase presence of same.

It is to be understood, of course, that the design, material and construction of the aerosol device, as well as the specification of the metered valve member suitable for dispensing the aerosol fragrance composition would be apparent to one of ordinary skill in the art without undue experimentation and should be considered as within the scope of this disclosure.

While only certain embodiments have been set forth, alternative embodiments and various modifications will be apparent from the above description to those skilled in the art. These and other alternatives are considered equivalents and within the spirit and scope of this disclosure and the appended claims.

What is claimed:

1. An aerosol fragrance composition for air freshening comprising:
from about 8 to about 20 wt % fragrance;
from about 25 to about 50 wt % organic solvent; and
from about 30 to about 67 wt % propellant,
wherein the aerosol composition is a single-phase composition at room temperature, and
wherein the Fragrance Strength of the composition dispensed through a 60 μL metered aerosol device is no less than 80% of the Fragrance Strength of the composition dispensed through a 120 μL metered aerosol device

2. The aerosol composition of claim 1 wherein the fragrance is included in the composition at a concentration of about 8 to about 10 wt %.

3. The aerosol composition of claim 1 wherein the organic solvent is selected from the group consisting of monohydric alcohols, glycols, acetone, C5-C9 hydrocarbon solvents, esters, diesters, and mixtures thereof.

4. The aerosol composition of claim 1 wherein the organic solvent is ethanol

5. The aerosol composition of claim 1 wherein the propellant is selected from the group consisting of propane, isobutane, n-butane, dimethyl ether, HFC-152a, and mixtures thereof.

6. The aerosol composition of claim 1 wherein the Fragrance Acceptance of the composition dispensed through a 60 μL metered aerosol device is no less than 80% of the Fragrance Acceptance of the composition dispensed through a 120 μL metered aerosol device.

7. The aerosol composition of claim 1 wherein the Fragrance Acceptance of the composition within 30 minutes after the composition is dispensed is more than 30%.

8. An aerosol fragrance composition for air freshening comprising:
from about 8 to about 20 wt % fragrance;
from about 25 to about 50 wt % organic solvent; and
from about 30 to about 67 wt % propellant,
wherein the aerosol composition is a single-phase composition at room temperature, and
wherein the Fragrance Acceptance of the composition dispensed through a 60 μL metered aerosol device is no less than 80% of the Fragrance Acceptance of the composition dispensed through a 120 μL metered aerosol device.

9. The aerosol composition of claim 8 wherein the organic solvent is selected from the group consisting of monohydric alcohols, glycols, acetone, C5-C9 hydrocarbon solvents, esters, diesters, and mixtures thereof.

10. The aerosol composition of claim 8 wherein the solvent is ethanol.

11. The aerosol composition of claim 8 wherein the propellant is a hydrocarbon propellant selected from the group consisting of propane, isobutane, n-butane, dimethyl ether, HFC-152a, and mixtures thereof.

12. The aerosol composition of claim 8 wherein the fragrance is included in the composition at a concentration of about 8 to about 10 wt %.

13. The aerosol composition of claim 8 wherein the Fragrance Acceptance of the composition within 30 minutes after the composition is dispensed is more than 30%.

14. An air-freshening aerosol package, comprising:
an aerosol device having a metered valve member; and
an aerosol composition enclosed in the aerosol device, the aerosol composition comprising from about 8 to about 20 wt % fragrance; from about 25 to about 50 wt % organic solvent; and from about 30 to about 67 wt % propellant,
wherein the aerosol device dispenses a metered dose of the aerosol composition to the air,
wherein the aerosol composition is a single-phase composition at room temperature, and
wherein the Fragrance Strength of the composition dispensed through a 60 μL metered aerosol device is no less than 80% of the Fragrance Strength of the composition dispensed through a 120 μL metered aerosol device.

15. The aerosol package of claim 14 wherein the organic solvent is selected from the group consisting of monohydric alcohols, glycols, acetone, C5-C9 hydrocarbon solvents, esters, diesters, and mixtures thereof.

16. The aerosol package of claim 14 wherein the organic solvent is ethanol.

17. The aerosol package of claim 14 wherein the propellant is selected from the group consisting of propane, isobutane, n-butane, dimethyl ether, HFC-152a, and mixtures thereof.

18. The aerosol package of claim 14 wherein the Fragrance Acceptance of the composition dispensed through a 60 μL metered aerosol device is no less than 80% of the Fragrance Acceptance of the composition dispensed through a 120 μL metered aerosol device.

19. The aerosol package of claim 14 wherein the metered dose ranges from about 20 to about 185 μL.

20. The aerosol package of claim 14 wherein the metered dose is about 60 μL.
